(19) United States
(12) Patent Application Publication
(10) Pub. No.: US 2022/0146077 A1
(43) Pub. Date: May 12, 2022

(54) FIELD LIGHTS AND RANGE FINDERS FOR RADIATION GENERATING DEVICES

(71) Applicant: Saunders Instruments, Inc., Bainbridge Island, WA (US)

(72) Inventors: Rick SAUNDERS, Bainbridge Island, WA (US); Malcolm SAUNDERS, Bainbridge Island, WA (US)

(21) Appl. No.: 17/454,137

(22) Filed: Nov. 9, 2021

Related U.S. Application Data
(60) Provisional application No. 63/111,897, filed on Nov. 10, 2020.
(60) Provisional application No. 63/185,730, filed on May 7, 2021.

(51) Int. Cl. F21V 11/08; G01C 3/02

(52) U.S. Cl. CPC F21V 11/08; F21W 2131/205; G01C 3/02

(57) ABSTRACT

Light devices, such as field lights or range finders, used in radiation generating devices, such as linear accelerators. The radiation generating devices can be used for, but are not limited to, medical treatment applications. Improved configurations for the light devices are described. The light devices may include one or more light emitting diodes (LEDs) as a light source which can be used to replace an existing light source, for example a light source that uses a halogen lamp. For example, a light device for a radiation generating device can include at least one light emitting diode having an illumination axis and at least one optical element having an optical axis, where the illumination axis is offset from the optical axis.

### FIELD

This technical disclosure relates to radiation generating devices including, but not limited to, those used in medical treatment applications that use a light device such as a field light and/or a range finder.

### BACKGROUND

Radiation generating devices used in medical treatment use field lights to project a pattern of light onto a patient to indicate the approximate pattern of radiation to be projected onto the patient, and range finders that project one or more indicators onto the patient to indicate a distance between the radiation generating device and the patient. The light sources for the field lights and the range finder may wear out over time and need replacement. The use of replaceable light sources in field lights and range finders for medical treatment radiation generating devices are known from U.S. Pat. Nos. 8,729,506, 9,463,075, and 10,285,771.

### SUMMARY

Light devices, such as field lights or range finders, used in radiation generating devices, such as linear accelerators, are described. The radiation generating devices can be used for, but are not limited to, medical treatment applications. Improved configurations for the light devices are described. The light devices may include one or more light emitting diodes (LEDs) as a light source which can be used to replace an existing light source, for example a light source that uses a halogen lamp.

In one embodiment, a light device for a radiation generating device can include at least one light emitting diode having an illumination axis and at least one optical element having an optical axis, where the illumination axis is offset from the optical axis.

In another embodiment, a light device for a radiation generating device can include at least one light emitting diode, at least one optical element having an optical axis, and a mirror between the at least one light emitting diode and the at least one optical element. The mirror reflects light emitted by the at least one light emitting diode through the at least one optical element along the optical axis.

In another embodiment, a replaceable light source for a radiation generating device is described. The replaceable light source can include a housing having a mounting mechanism for removably mounting the housing to the radiation generating device. At least one light emitting diode is mounted to the housing, where the at least one light emitting diode has an illumination axis. When the housing is mounted to the radiation generating device, the illumination axis is perpendicular to an optical axis of the radiation generating device or laterally offset from the optical axis.

In still another embodiment, a light device for a radiation generating device can include a light assembly that has an array of light emitting diodes. In addition, an optical assembly is provided that has an optical axis, and the optical assembly includes at least one of the following: at least one baffle; at least one reticle; at least one projection lens; or at least one condenser lens.

In still another embodiment, a light device for a radiation generating device can include a light assembly that has a light support that is mounted in the radiation generating device so as to be moveable between a first use position for providing illumination and a second use position for providing illumination. A first light emitting diode is mounted on the light support and a second light emitting diode mounted on the light support at a position spaced from the first light emitting diode, wherein the first light emitting diode provides illumination when the light support is at the first use position and the second light emitting diode provides illumination when the light support is at the second use position.

In still another embodiment, a light device for a radiation generating device can include a housing having an aperture, and at least one light emitting diode in the housing and that emits light that exits through the aperture, where the light device is devoid of optical components.

### DRAWINGS

FIG. 1 schematically depicts a radiation generating device configured for medical treatment and that uses a field light and a range finder.

FIG. 2 is a cross-sectional side view of an embodiment of a light device in the form of a range finder with a replaceable light source.

FIG. 3 is a top perspective view of a light housing of the replaceable light source of FIG. 2.

FIG. 4 is a bottom perspective view of the light housing of FIG. 3.

FIG. 5 is an exploded top perspective view of another embodiment of a light housing holder and light housing for a ranger finder.

FIG. 6 illustrates the light housing of FIG. 5 installed in the light housing holder.

FIG. 7 is a cross-sectional side view of another embodiment of a light device in the form of a range finder with a removable light source.

FIGS. 8A and 8B illustrate another embodiment of a light device in the form of a range finder that uses an array of LEDs.

FIG. 8C and 8D illustrate another embodiment of a light device that uses an array of LEDs.

FIG. 9 is a cross-sectional side view of another embodiment of a light device in the form of a range finder.

FIG. 10 is a cross-sectional side view of another embodiment of a light device in the form of a range finder.

FIG. 11 is a cross-sectional side view of another embodiment of a light device in the form of a range finder.

FIG. 12A is a cross-sectional side view of an embodiment of a light device in the form of a field light with a replaceable light source.

FIG. 12B is a perspective view of the replaceable light source of FIG. 12A.

FIG. 13 is a perspective view of another embodiment of a light device in the form of a field light without any optical components.

FIG. 14 is a cross-sectional side view of the field light of FIG. 13 taken along line 14-14.

FIG. 15 illustrates an assembly for a field light that uses multiple LED assemblies and the assembly can be moved from a first position using the first LED assembly to a second position using the second LED assembly.

FIG. 16 illustrates another example of a light housing of a light source similar to the light housings in FIGS. 2-6 but with an LED array.

### DETAILED DESCRIPTION

The following is a detailed description of embodiments of light devices used in radiation generating devices, such as linear accelerators. The light devices can be field lights or range finders. The radiation generating devices can be used for medical/therapeutic treatment applications. For example, in the case of linear accelerators, the radiation generating device can be used for external beam radiation treatments of cancer patients using high-energy x-rays or electrons, for example in Intensity-Modulated Radiation Therapy (IMRT), Volumetric Modulated Arc Therapy (VMAT), Image Guided Radiation Therapy (IGRT), Stereotactic Radiosurgery (SRS), and Stereotactic Body Radio Therapy (SBRT), and other treatment applications. However, the light devices described herein can be used in any technology, in the medical field or outside the medical field, that uses light devices such as field lights and/or range finders.

The devices described herein include a light source having one or more LEDs. In some embodiments the light source may be intended to be fixed or permanent (i.e. not replaceable separately from the other elements of the light device). In some embodiments the light source may be intended to be replaceable separately from the other elements of the light device.

A replaceable light source that is for use with a light device such as a field light or a range finder is also described. The light source uses at least one LED and can replace an existing light source that uses a halogen lamp or the light source can replace an existing light source that uses an LED.

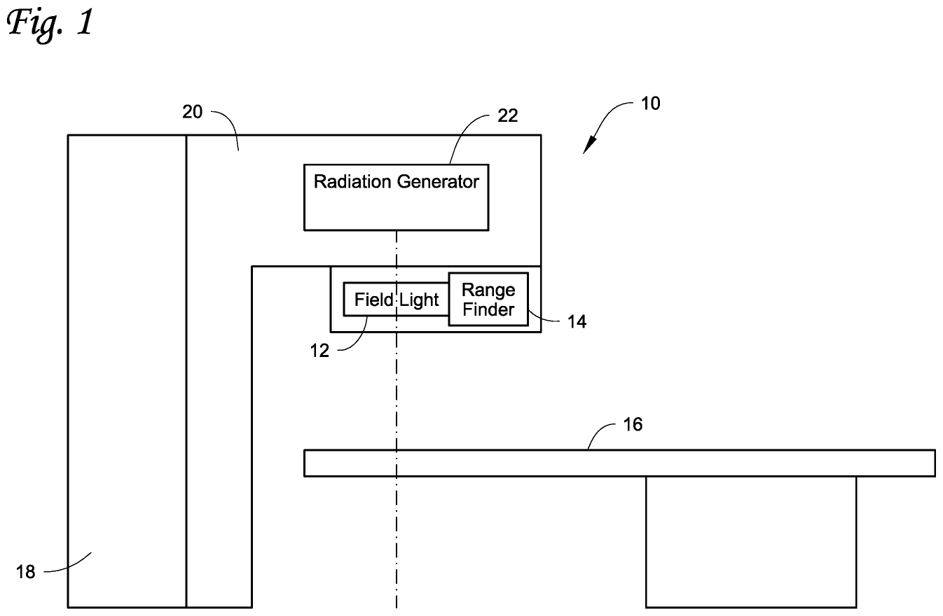

With reference initially to FIG. 1, an example of a radiation generating device 10 that uses light sources such as a field light 12 and a range finder 14 is illustrated. In this example, the radiation generating device 10 is configured for medical treatment of a patient (not shown) laying on or otherwise disposed on a table 16. The general construction and operation of the radiation generating device 10, except for the specific constructions of the field lights 12 and the range finders 14 described herein, is conventional and well known in the art. In this example, the device 10 generally includes a support structure 18 and a gantry 20. The device 10 further includes a radiation generator 22 that generates radiation that is directed onto the patient for use in medical/therapeutic treatment of the patient. The radiation generator 22 can be or can include, for example, a linear accelerator. Other constructions of the radiation generating device 10 are possible. Further details on the general construction and operation of a radiation generating device can be found in U.S. Pat. Nos. 8,729,506, 9,463,075, and 10,285,771.

The following description describes a number of examples of light devices in the form of field lights and range finders. Many of the light devices, whether a range finder or a field light, described herein can share common features such as a light source having one or more LEDs, and an optical assembly with one or more optical elements through which light from the light source is directed. In the field lights, the optical assembly is configured to project a pattern of light onto a patient to indicate the approximate pattern of radiation to be projected onto the patient. In the range finders, the optical assembly includes a reticle or other device for projecting one or more indicators onto the patient to indicate a distance between the radiation generating device and the patient. However, not all embodiments described herein require an optical assembly. In addition, any of the features of the range finders described herein can be implemented on the field lights, and any of the features of the field lights described herein can be implemented on the ranger finders.

The optical assemblies described herein can have one or more condenser lenses, a baffle, a reticle, and one or more projection lenses. The construction and function of these optical elements are well known in the art of radiation generating devices that use light sources. A reticle is a disk made of glass or other light transparent material having a pattern, such as lines and characters, etched or otherwise provided thereon that is projected onto the table 16 or onto a person laying on the table 16.

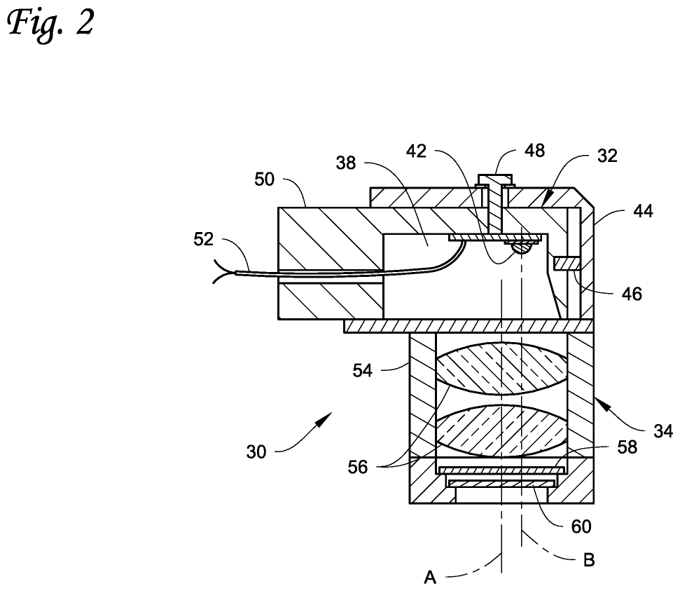

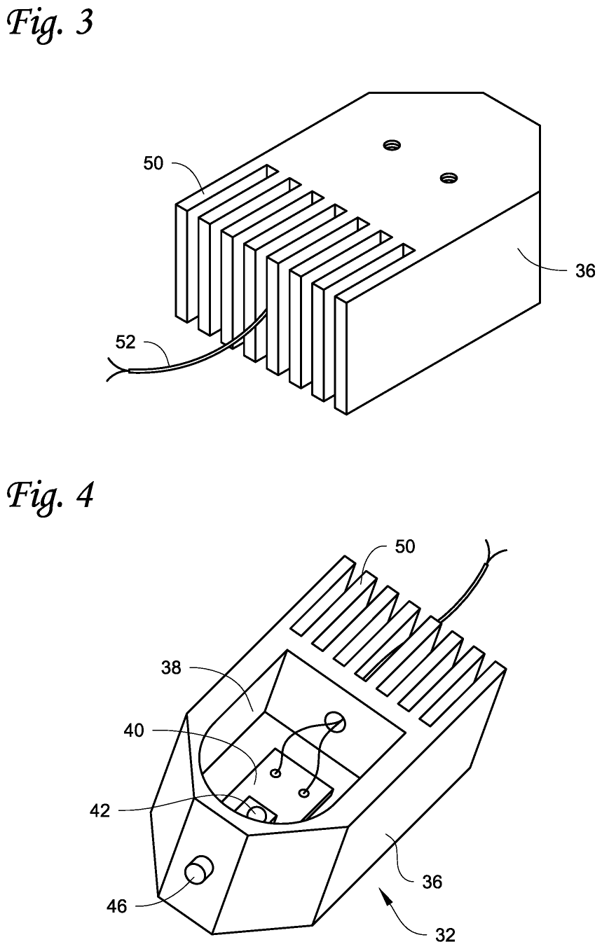

With reference to FIGS. 2-4, an example of a light device in the form of a range finder 30 is illustrated. The range finder 30 includes a light source 32 and an optical assembly 34. In this example, the light source 32 is replaceable to allow easy replacement of an existing light source such as a halogen light source or replace an LED light source. The light source 32 includes a light housing 36 having a recessed area 38 on a bottom side thereof in which is mounted a circuit board 40 containing at least one LED 42 that provides illumination. The housing 36 is removably held in a light housing holder 44. One end of the housing 36 includes a locator pin 46 that when fully installed abuts against a surface inside the holder 44, and one or more mechanical fasteners 48, such as screws, detachably secure the housing 36 to the holder 44. The opposite end of the housing 36 can include a plurality of heat exchange fins 50 that help to dissipate heat generated by the LED(s) 42. Power for the LED(s) 42 can be provided by a power cable 52.

With continued reference to FIGS. 2-4, the optical assembly 34 includes an optical assembly housing 54 that houses one or more optical elements such as one or more condenser lenses 56, a baffle plate 58, and a reticle 60. The housing 54 may be integral with or separate from the holder 44. As depicted in FIG. 2, the optical assembly 34 has an optical axis A. In addition, when the light source 32 is properly mounted in the holder 44, the LED 42 has an illumination axis B that is laterally offset from the optical axis A. The lateral offset between the axes A and B can be in any direction (e.g. left or right, forward or rearward, and any angle therebetween when viewing FIG. 2). Offsetting the illumination axis B from the optical axis A puts more light at a specific location, for example at the small end of the reticle image. Preferably, the axes A and B are parallel to one another. However, the axes A and B can be angled relative to one another such that they are laterally displaced from each other in the range finder 30 so that they would intersect one another outside the range finder 30.

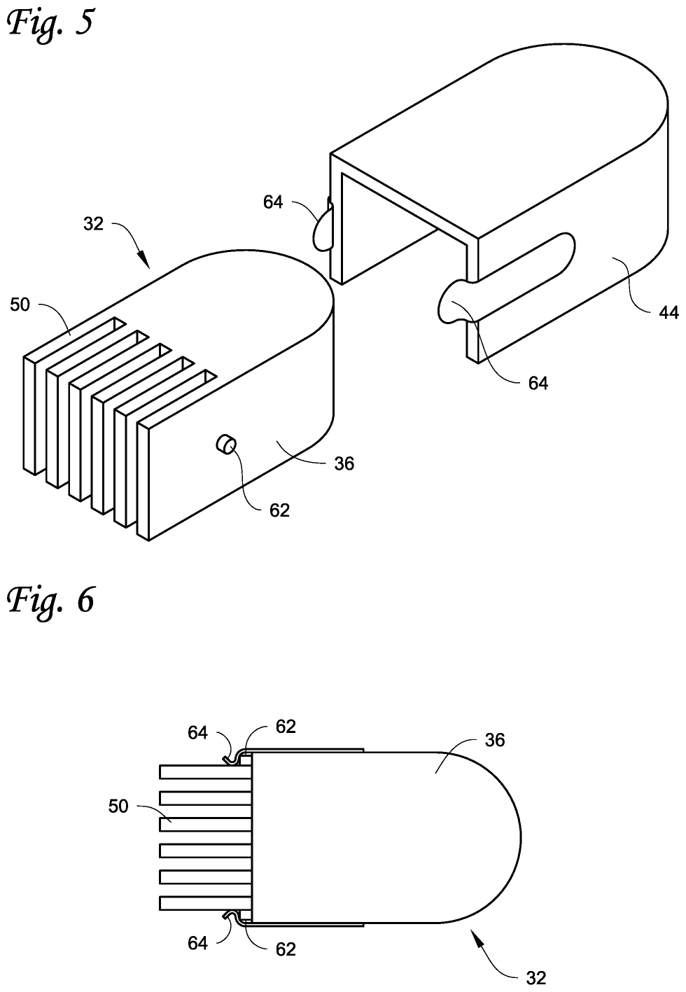

FIGS. 5-6 illustrate another example of the light source 32 that can be used with the range finder 30 of FIGS. 2-4. In FIGS. 5-6, elements that are the same as or similar to elements in FIGS. 2-4 are referenced using the same reference numerals. In this example, instead of using mechanical fasteners, such as screws, to secure the housing 36 to the holder 44, the housing 36 includes a pair of pins 62 and the holder 44 includes a pair of pin engagement springs 64 that snap fit engage with the pins 62 as depicted in FIG. 6 to secure the light source 32 in the holder 44. The light source 32 can be otherwise similar to the light source in FIGS. 2-4 including the recessed area 38 on a bottom side thereof in which is mounted the circuit board 40 containing the LED(s) 42 which, when mounted in the holder 44, will have its illumination axis offset from the optical axis of the optical assembly.

FIG. 16 illustrates another example of a light source 32 similar to the light sources 32 in FIGS. 2-6. Elements in FIG. 16 that are similar to elements in FIGS. 2-6 are referenced using the same reference numerals. In FIG. 16, the light source 32 includes the light housing 36 having the recessed area 38 on a bottom side thereof, the heat exchange fins 50, and the pins 62 for mounting the light source 32 in the light housing holder 44 in FIGS. 5-6. The light source 32 in FIG. 16 differs from the light sources in FIGS. 2-6 in that an LED array 66 is disposed in the recessed area 38. The LED array 66 includes a plurality of individual LEDs 67. The LED array 66 can have any shape such as circular (shown in FIG. 16), square, rectangular, oval, triangular, and other shapes. The LEDs 67 in the array 66 can be arranged in any pattern. The array 66 is depicted as being disposed on a substrate 68, for example a rectangular substrate.

The use of an LED array, such as the array 66, provides a number of advantages over using a single LED or other light emitting element. For example, the array can be attached directly to a housing or heat sink, eliminating the need for a separate printed circuit board. The array creates an extended light source that can illuminate the entire area of the reticle more evenly than a single light emitting element can which creates a projected image with more even brightness. In addition, because the array acts as an extended light source, it does not need to be located as accurately in the plane perpendicular to the optical axis. The illumination axis B (see FIG. 2) can be offset from the optical axis A, but there is more tolerance using the array than with the light source 32 using a single light emitting element. The shape of the array can be tailored to illuminate only part of the reticle or other object that is to be illuminated, thereby producing less waste heat. The array may be fastened to the light housing 36 in any suitable manner, for example using an adhesive such as, but not limited to, a thermally conductive adhesive, or using one or more mechanical fasteners such as screws, mechanical clips or retainers. The array 66 is shown as being used with the removable light housing 36. However, the array 66 can be used with a non-removable light housing.

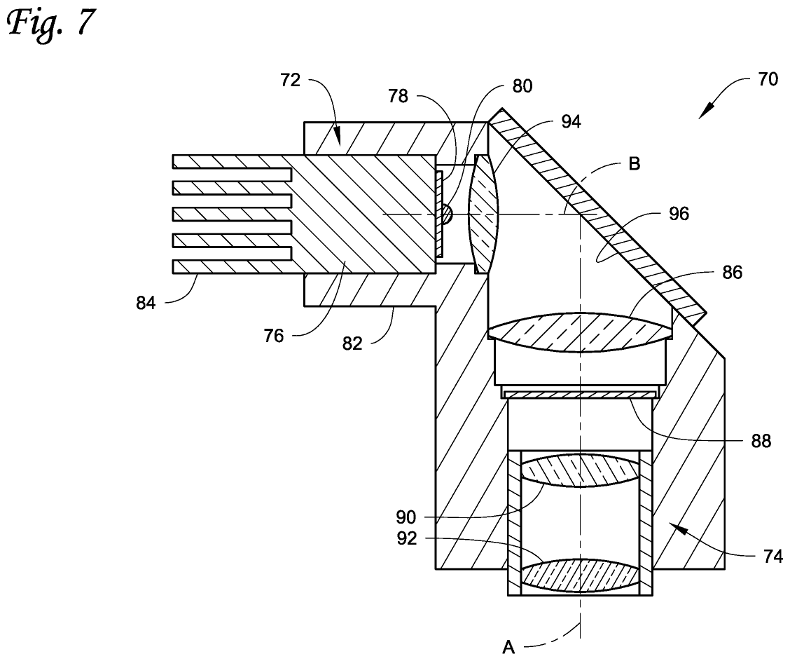

With reference to FIG. 7, another example of a light device in the form of a range finder 70 is illustrated. The range finder 70 includes a light source 72 and an optical assembly 74. In this example, the light source 72 is replaceable to allow easy replacement of an existing light source such as a halogen light source or replace an LED light source. The light source 72 includes a light housing 76 having a circuit board 78 mounted thereon that includes at least one LED 80 that provides illumination. The housing 76 is removably held in a light housing holder 82 in any suitable manner, for example one of the securing mechanisms described in FIGS. 2-6 can be used. The end of the housing 76 opposite the LED(s) 80 can include a plurality of heat exchange fins 84 that help to dissipate heat generated by the LED(s) 80. Power for the LED(s) 80 can be provided by a power cable electrically connected to the LED(s) 80 via the circuit board 78.

With continued reference to FIG. 7, the optical assembly 74 can include a condenser lens 86, a reticle 88, and a projection lens assembly that can include, for example, a pair of projection lenses 90, 92. In addition, a second condenser lens 94 can be mounted in the light housing holder 82 in front of the LED(s) 80. In this embodiment, the illumination axis B of the LED(s) 80 is oriented 90 degrees from (i.e. offset from) and perpendicular to the optical axis A. A mirror 96 can be provided to deflect the light from the LED(s) 80 ninety degrees and onto the optical axis A. The mirror 96 can be considered part of the optical assembly 74 or separate from the optical assembly 74.

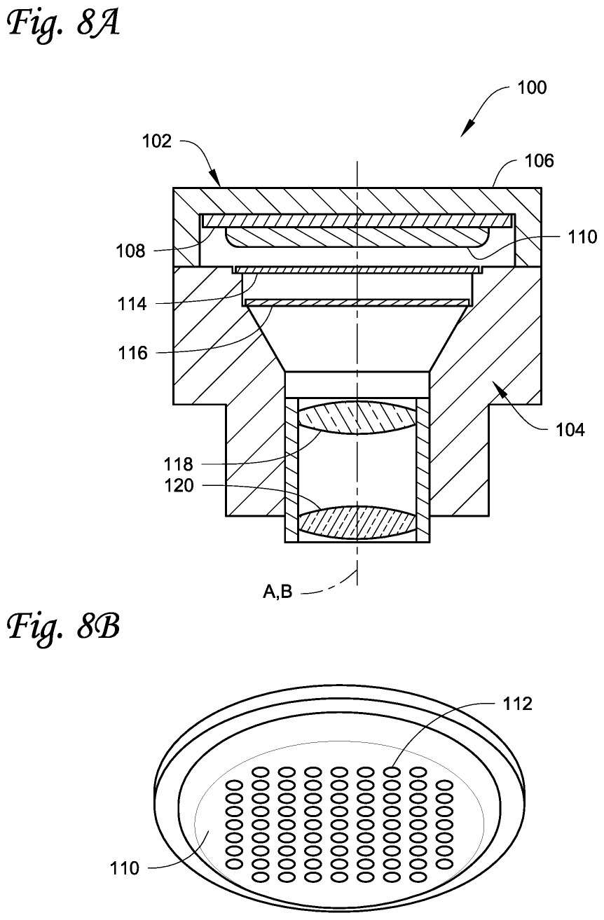

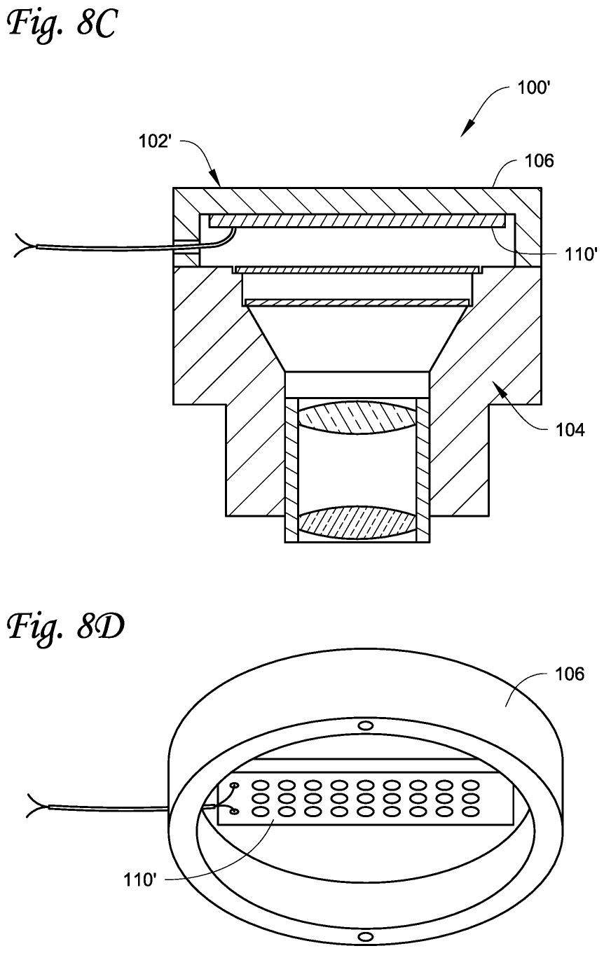

With reference to FIGS. 8A and 8B, another example of a light device in the form of a range finder 100 is illustrated. The range finder 100 includes a light source 102 and an optical assembly 104. In this example, the light source 102 may or may not be replaceable. The light source 102 includes a light housing 106 having a circuit board 108 mounted therein, and an LED array 110 mounted on the circuit board 108. The array 110 approximates a large surface light source and is positioned to fill the reticle with light. The array 110 may or may not be considered as having an illumination axis. The LED array 110 comprises a plurality of individual LEDs 112, for example arranged in a plurality of rows and columns with the individual LEDs 112 disposed close to one another. In another embodiment, the LEDs 112 can be arranged randomly, or disposed in any arrangement that provides the desired illumination. In one non-limiting embodiment, the LED array 110 can be a chip-on-board array. The array 110 can be circular as depicted, rectangular, square, oval, triangular, or have any other shape. Similarly, the LEDs 112 can be arranged into a circular arrangement, rectangular, square, oval, triangular, or any other arrangement. In addition, although FIG. 8A shows the circuit board 108 between the array 110 and the housing 106, the light source 102 does not necessarily need to have the circuit board 108 between the array 110 and the housing 106.

The optical assembly 104 includes a baffle 114, a reticle 116 and a projection lens assembly having a pair of projection lenses 118, 120 defining the optical axis A. In this example, the range finder 100 is devoid of condenser lenses, i.e. no condenser lenses are necessary. In addition, the baffle 114 is optional. In the illustrated example, the array 110 is depicted as being centered on the optical axis A. However, the array 110 can be positioned or the LEDs 112 disposed on the array 110 in such a manner that the array 110 is not centered on the optical axis A.

FIGS. 8C and 8D illustrate another embodiment of a light device, for example in the form of a range finder 100′. Elements in FIGS. 8C and 8D that are similar to elements in Figures 8A and 8B are referenced using the same reference numerals. The light device includes a light source 102′ and the optical assembly 104. In this example, the light source 102′ may or may not be replaceable. The light source 102′ includes a light housing 106 and an LED array 110′. In this example, the array 110′ is configured as a rectangular array with a plurality of individual LEDs 112, for example arranged in at least two longitudinal rows. However, the array can have configurations other than rectangular, such as circular, square, oval, triangular and the like, and the LEDs 112 can be arranged randomly, or disposed in any arrangement that provides the desired illumination. A circuit board may or may not be provided between the array 110′ and the housing 106.

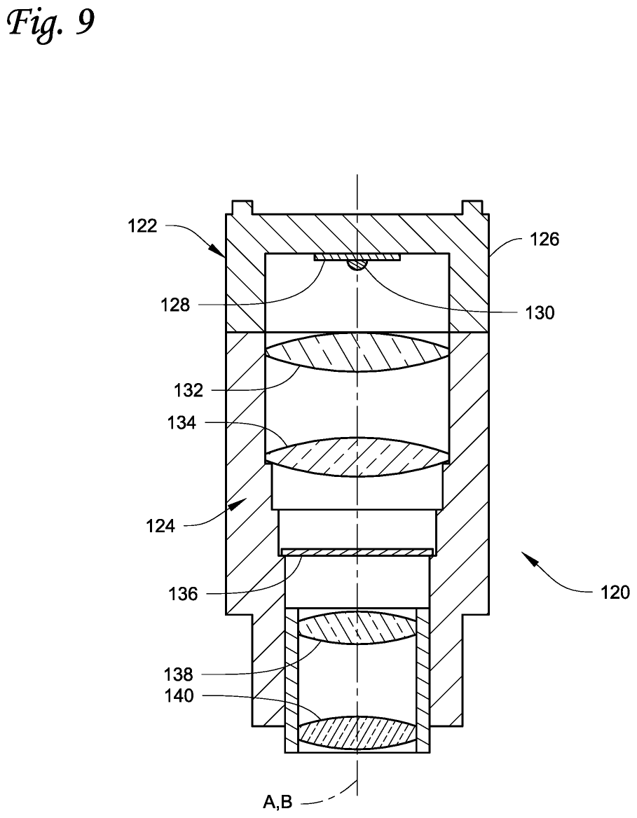

With reference to FIG. 9, another example of a light device in the form of a range finder 120 is illustrated. The range finder 120 includes a light source 122 and an optical assembly 124. The light source 122 includes a light housing 126 having a circuit board 128 mounted therein, and at least one LED 130 mounted on the circuit board 128. The LED 130 is positioned so that the illumination axis B coincides with the optical axis A. The optical assembly 124 is illustrated as including condenser lenses 132, 134, a reticle 136 and projection lenses 138, 140.

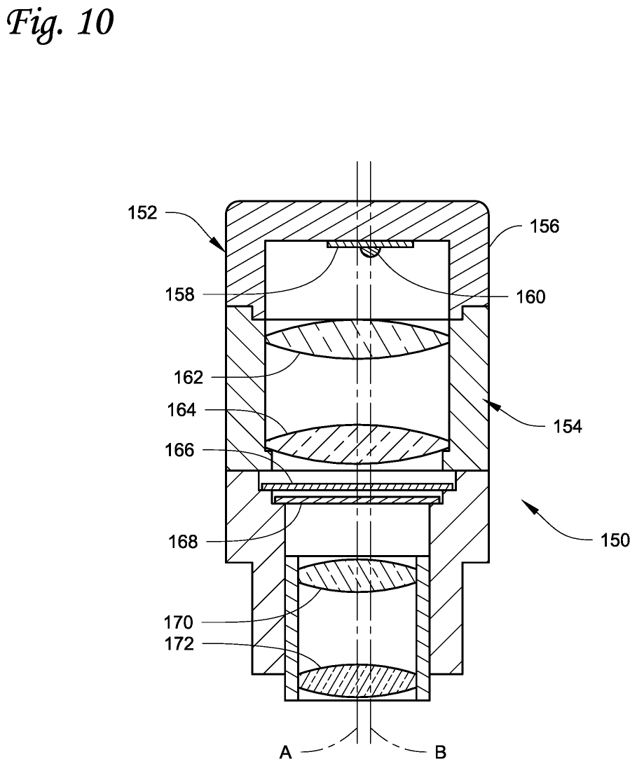

With reference to FIG. 10, another example of a light device in the form of a range finder 150 is illustrated. The range finder 150 includes a light source 152 and an optical assembly 154. In this example, the light source 152 may or may not be replaceable. The light source 152 includes a light housing 156 having a circuit board 158 mounted therein, and at least one LED 160 mounted on the circuit board 158. In this example, the LED 160 is positioned so that the illumination axis B thereof is offset from, for example parallel to, the optical axis A. For example, the illumination axis B can be offset in order to put more light on the small end (i.e. the end with the smallest lines and characters) of the reticle of the optical assembly 154 which typically needs more light because it is projected farther than light from the larger end of the reticle. The optical assembly 154 is illustrated as including condenser lenses 162, 164, a baffle 166, a reticle 168, and projection lenses 170, 172.

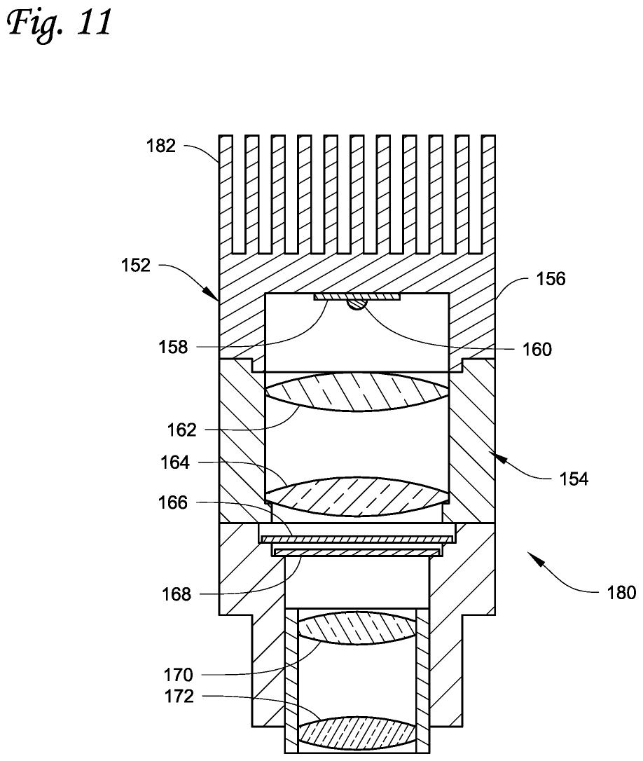

FIG. 11 illustrates a light device in the form of a range finder 180 that is similar to the range finder 150 in FIG. 10, and elements that are the same as or similar to elements in FIG. 10 are referenced using the same reference numerals. In this embodiment, the range finder 180 includes heat exchange fins 182 on the light housing 156 for dissipating heat.

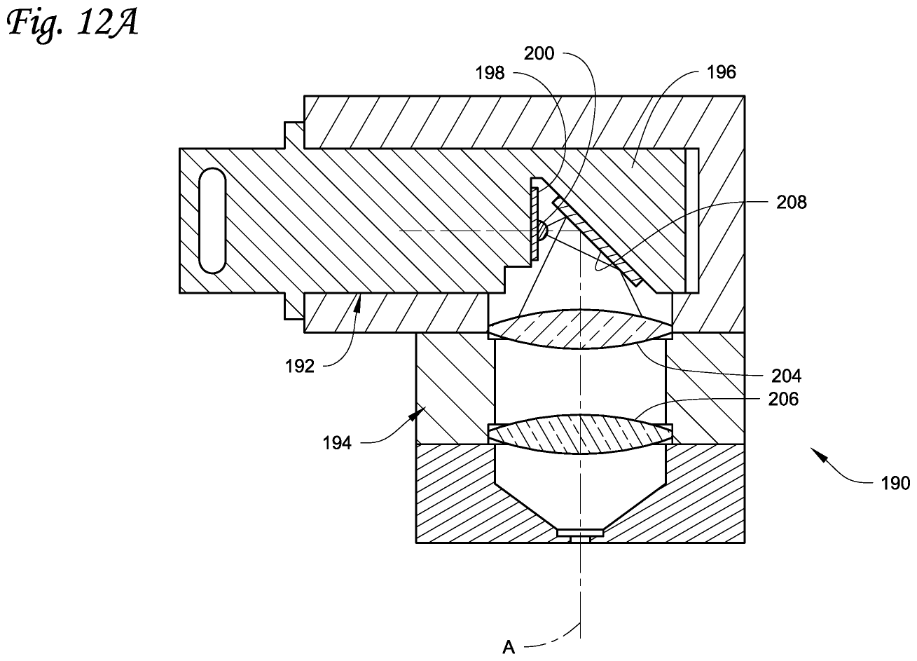

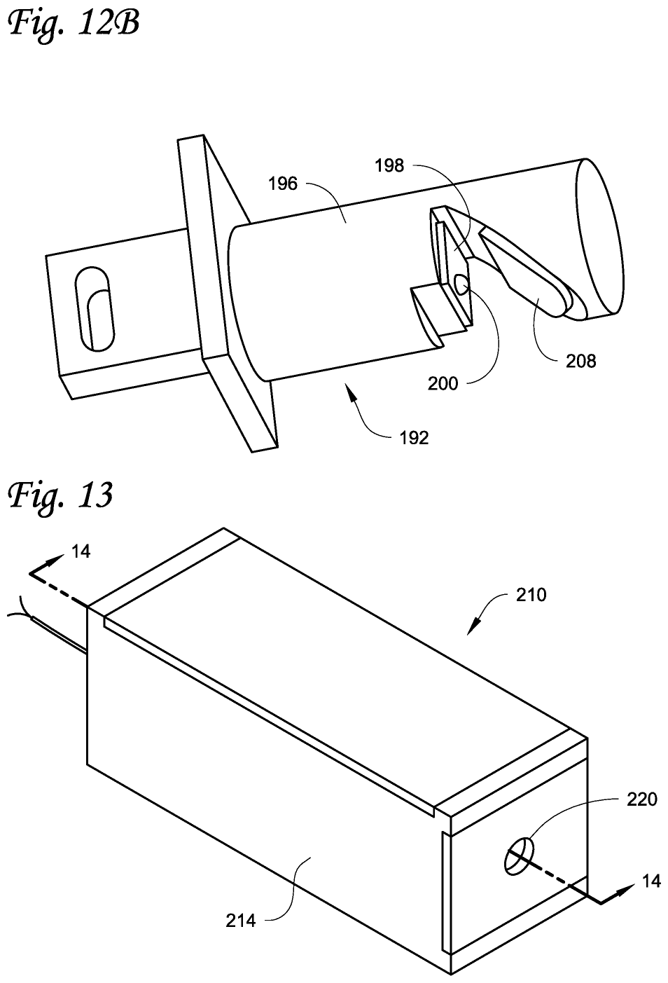

With reference to FIGS. 12A and 12B, another example of a light device in the form of a field light 190 is illustrated. The field light 190 includes a light source 192 and an optical assembly 194. In this example, the light source 192 is configured to be replaceable whereby the light source 192 is detachably mounted to the field light 190. The light source 192 includes a light housing 196 having a circuit board 198 mounted thereon, and at least one LED 200 mounted on the circuit board 198. A mirror 208 is also disposed on or in the light housing 196 and is removable with the light housing 196. The end of the housing 196 opposite the LED(s) 200 can optionally include a plurality of heat exchange fins that help to dissipate heat generated by the LED(s) 200. Power for the LED(s) 200 can be provided by a power cable electrically connected to the LED(s) 200 via the circuit board 198.

With continued reference to FIGS. 12A and 12B, the optical assembly 194 can include a condenser lens assembly having a pair of condenser lenses 204, 206. In this embodiment, the illumination axis of the LED(s) 200 is oriented 90 degrees from (i.e. offset from) and perpendicular to the optical axis A. The mirror 208 deflects the light from the LED(s) 200 ninety degrees and onto the optical axis A. In some embodiments, the mirror 208 can be separate from the light source 192, for example the mirror 208 can be part of the optical assembly 194.

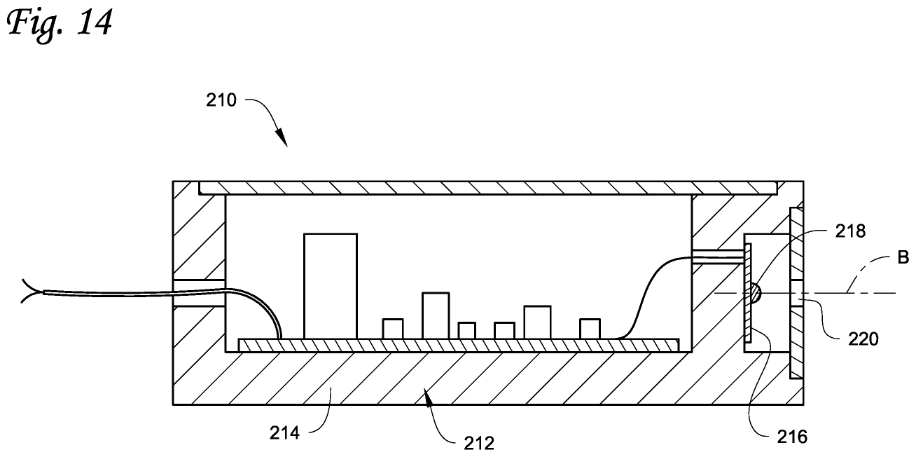

FIGS. 13-14 illustrate another light device in the form of a field light 210 without any optical components. In this example, the field light 210 includes a light source 212 that has a light housing 214 with a circuit board 216 mounted therein, and at least one LED 218 mounted on the circuit board 216. Light from the LED(s) 218 exits the housing 214 through a hole 220 along the illumination axis B. An LED driver to drive the LED 218 is inside the housing 214. The field light 210 is devoid of any lenses or other optical components, and the entire field light 210 can be replaced as a unit when necessary.

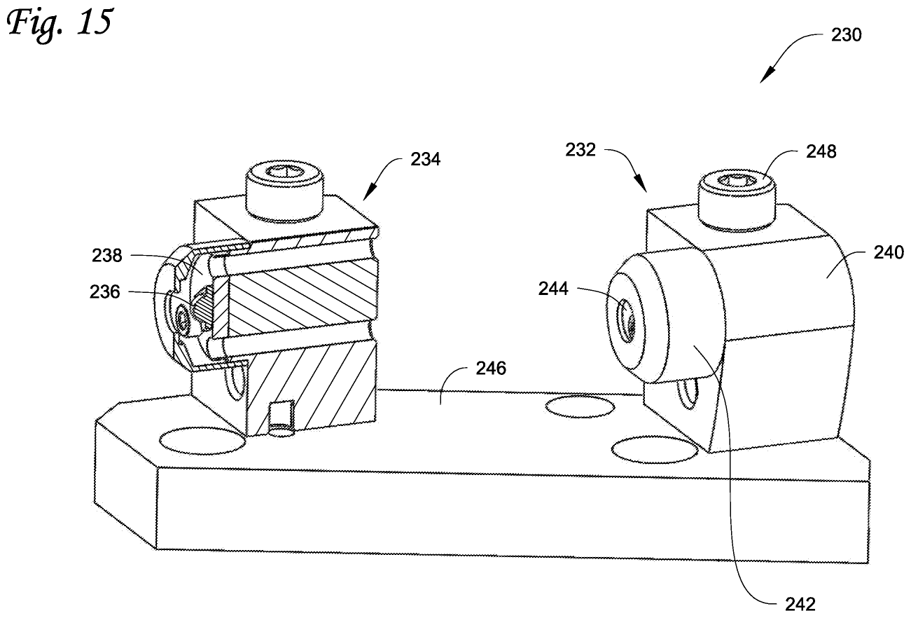

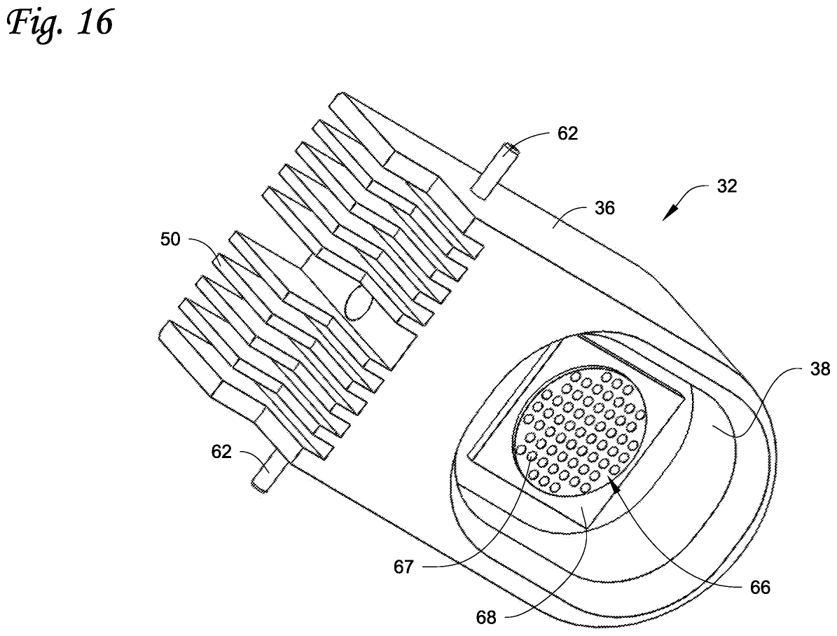

FIG. 15 illustrates another light device in the form of a field light 230. The field light 230 is illustrated without any optical components, but in some embodiments the field light 230 could include optical components. This example illustrates a pair of light assemblies 232, 234 each of which can individually be considered a field light. Each light assembly 232, 234 includes an LED 236 mounted on a circuit board 238, and a light housing 240. A cap 242 is mounted to the light housings 240 and each cap 242 includes an apertures 244 through which light is emitted. In some embodiments, the LEDs 236 of the light assemblies 232, 234 can be identical and output the same lumen value of light. In other embodiments, one of the LEDs 236 can output light with a first lumen value and/or a first color, while the second LED 236 can output light with a second lumen value different from the first lumen value and/or a second color different from the first color. Each light assembly 232, 234 can be removably attached to a light support 246 via mechanical fasteners such as screws 248 that detachably affix the light housings 240 to the support 246. This permits the entire light assembly 232, 234 to be removed and replaced as needed. In other embodiments, the LEDs 236 can be replaced without replacing the entire light assembly 232, 234, for example by removing the cap 242, removing the circuit board 238 with the LED 236 thereon (or just removing the LED 236) and installing a new circuit board 238 with a new LED 236.

In the example of FIG. 15, the field light 230 is mounted so as to be movable between a first use position for providing illumination using the LED 236 of the first light assembly 232 and to a second use position for providing illumination using the LED 236 of the second light assembly 234. Radiation generating devices that include two lamps that are mounted so to be movable between two use positions is known. However, those devices use halogen lamps and only the lamps are replaced. In contrast, any one of the individual light assemblies 232, 234 can be replaced when one or more of the LEDs 236 needs replacement or if a change in the light output is desired.

Additional embodiments can include the following:

A light device for a radiation generating device can include at least one light emitting diode; at least one optical element having an optical axis; and a mirror between the at least one light emitting diode and the at least one optical element, the mirror reflecting light emitted by the at least one light emitting diode through the at least one optical element along the optical axis.

A replaceable light source for a radiation generating device having an optical axis, including a housing having a mounting mechanism for removably mounting the housing to the radiation generating device; and at least one light emitting diode mounted to the housing, the at least one light emitting diode having an illumination axis; wherein when the housing is mounted to the radiation generating device, the illumination axis is perpendicular to the optical axis or laterally offset from the optical axis.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A light device for a radiation generating device, comprising:
at least one light emitting diode having an illumination axis;
at least one optical element having an optical axis; and
the illumination axis is offset from the optical axis.

2. The light device of claim 1, wherein the light device is a field light or a range finder.

3. A light device for a radiation generating device, comprising:
a light assembly that includes an array of light emitting diodes, an optical assembly having an optical axis, the optical assembly includes at least one of the following:
a. at least one baffle;
b. at least one reticle;
c. at least one projection lens; and
d. at least one condenser lens.

4. The light device of claim 3, wherein the light device is a field light or a range finder.

5. A light device for a radiation generating device, comprising:
a light assembly that includes a light support that is mounted in the radiation generating device so as to be moveable between a first use position for providing illumination and a second use position for providing illumination;
a first light emitting diode mounted on the light support and a second light emitting diode mounted on the light support at a position spaced from the first light emitting diode, wherein the first light emitting diode provides illumination when the light support is at the first use position and the second light emitting diode provides illumination when the light support is at the second use position.

6. The light device of claim 5, wherein the light device is a field light or a range finder.

7. A light device for a radiation generating device, comprising:
a housing having an aperture;
at least one light emitting diode in the housing and that emits light that exits through the aperture; and
the light device is devoid of optical components.

8. The light device of claim 7, wherein the light device is a field light or a range finder.
